(19) United States
(12) Patent Application Publication
(10) Pub. No.: US 2009/0137656 A1
(43) Pub. Date: May 28, 2009

(54) Intermolecular Association Complex of a Carrier and of an Active Principle

(75) Inventors: Sabrina Consola, Toulouse (FR); Muriel Blanzat, Toulouse (FR); Isabelle Rico-Lattes, Auzielle (FR); Emile Perez, Colomiers (FR); Pascal Bordat, Mervilla (FR)

Correspondence Address: BIRCH STEWART KOLASCH & BIRCH, PO BOX 747, FALLS CHURCH, VA, 22040-0747, US

(73) Assignee: PIERRE FABRE DERMO-COSMETIQUE, Boulogne-Billancourt (FR)

(21) Appl. No.: 11/989,210

(22) PCT Filed: Jul. 21, 2006
(86) PCT No.: PCT/EP2006/064502

§ 371 Date: Jan. 22, 2008

(30) Foreign Application Priority Data
Jul. 22, 2005 (FR) 0507856

(51) Int. Cl. A61K 31/405; A61K 31/192; A61P 29/00; A61K 31/201

(52) U.S. Cl. 514/420; 514/570; 514/560

(57) ABSTRACT

The invention relates to an intermolecular association complex of formula (I) of an amphiphilic carrier and of an active principle Z-Y, in which; S, X n, R₁, R₂, R₃, Y, Z are as defined in the description.

Improving the stability of an active principle in order to reduce its secondary effects still now remains a highly important issue. In general, this stabilisation occurs on two levels. First, at the storage level where preservatives and antioxidants can be introduced, then during administration of the active principle. The latter will be administered by means of a vehicle to solubilise it and optionally target it to the action site. At the same time it will be protected from multiple degradations caused by the immune system.

Currently, there are numerous vectorization techniques for active principles.

These techniques use different strategies according to the nature of the active principle (hydrophilic or lipophilic), as well as of the targeted organ, of the dose administered and of the duration of administration. For example, the active principle could be encapsulated inside a vesicle of phospholipids or immobilised in microspheres of biodegradable polymer.

There are numerous advantages to delivery of active principles through the skin. The variable speeds of absorption and metabolisation associated with oral treatment are avoided, as are possible gastrointestinal irritations. Delivering the active principle transcutaneously also allows better control of its blood concentration.

However, the skin has a complex structure and the molecules administered transcutaneously or topically must first pass an initial barrier constituted by the stratum corneum prior to reaching the blood flow. The stratum corneum is constituted by a dense and highly keratinised layer of average thickness of 10-15 microns. The high degree of keratinisation, as well as compact assembly of the cells can constitute a barrier practically impermeable to the passage of an active principle. For the majority of drugs, the speed of permeabilisation through the skin, without addition of permeabilising additive, is extremely slow. Numerous additives can be used to boost the penetration speed of the active principle through the skin. The majority of compounds is administered at the same time as the drug (in certain cases the skin can be pre-treated with a permeabilisation agent) so as to increase the permeability of the stratum corneum and thus boost penetration of the active principle through the skin. The permeability of many therapeutic agents can be improved due to these permeabilisation agents. Several additives are capable of promoting the transport of active principles through the skin according to several mechanisms, whereof the most important are:
Extraction of lipids from the stratum corneum
Disorganisation of the structure of the lipidic bilayer
Displacement of the associated water
Delamination of the stratum corneum
Disorganisation of the corneous layer

Permeabilisation agents can be classified into different categories. Solvents such as alcohols, sulfoxide methyl alkyls and polyols, increase solubility, thus increasing cutaneous passage. Also, some solvents such as dimethylsulfoxyde (DMSO) or ethanol could extract lipids and make the stratum corneum more permeable. Oleic acid and isopropyl myristate are examples of permeabilisation agents which disorganise the corneous layer by interleaving in lipidic structures. This emollient effect thus increases the diffusion coefficient of the active principle. Likewise, ionic surfactants or DMSO interact with the keratin of corneocytes, which deploys the structure of the protein and boosts the diffusion coefficient.

The present invention describes an original strategy consisting in having the active principle participate actively in its own transport. The aim of this intermolecular association will be to protect, solubilise and convey the drug to the action site. For this, it is proposed to associate an active principle acid with a basic biocompatible amphiphilic molecule by simple acid/base electrostatic interaction. This association could be stabilised by interactions of hydrophobic type between the active principle and the amphiphilic molecule. In particular, this invention concerns applications in formulation, such as solubilisation, transport, protection and transcutaneous diffusion of an active principle. In fact, this amphiphilic molecule could also play the role of permeabilisation agent for transcutaneous transport.

The invention relates to association of a basic biocompatible transporter with an active principle comprising one or more acid functions. This intermolecular association leads to a novel amphiphilic type corresponding to formation of an acid/base pair associated by electrostatic interactions and stabilised by Van der Waals-type interactions between the hydrophobic parts of the two constituents. According to its concentration in water as well as the nature of the active principle (volume, hydrophobia), the amphiphilic complex thus formed by association results in a set of autoassembled structures such as micelles or vesicles. The resulting objects could also serve as auto-transport for the active principle.

The present invention therefore relates to an association complex formed between an amphiphilic molecule and an active principle.

The subject matter of the present invention is an intermolecular association complex of formula (I) of an amphiphilic transporter and an active principle ⁻Z-Y:

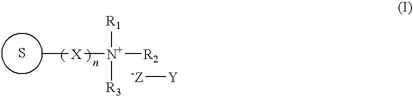

in which

S represents a residue of carbohydrates selected in the group comprising monosaccharides, disaccharides, polysaccharides, polyols, and combinations of these residues,

X represents a C1-C12 aliphatic residue selected from linear or branched alkyl, alkene, alkyne, or an ethylene oxide or propylene oxide unit having a degree of polymerisation of between 1 and 10, as well as all the combinations of these residues,

n=0 or 1

R1 represents H

R2, R3 independently represent a hydrogen atom or a linear or branched C1-C20 hydrocarbon chain or perfluorated, as well as all combinations of these substituents, and in which the active principle

⁻Z-Y

comprises a residue
Y carrying therapeutic or pro-therapeutic activity, selected from the group comprising anti-inflammatories, antibiotics, polyunsaturated fatty chain, vitamins or pro-vitamins
   and a residue
Z acid selected from the group comprising the carboxylates, sulphates, sulphonates, phosphates, phosphonates or phosphinates.

The present invention also relates to using a complex such as defined hereinabove to protect, solubilise and/or convey an active principle.

The invention also relates to using a complex such as defined hereinabove for producing a drug for topical or transcutaneous administration.

The transporter is selected from biocompatible amphiphilic molecules having one or more basic functions.

According to the present invention the amphiphilic transporter will be selected from derivatives of carbon hydrates having one or more hydrophobic chains, as well as one or more basic functions likely to interact electrostatically with the acid active principle.

This amphiphilic transporter responds to the general formula (II):

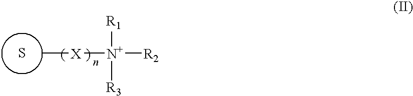

in which

S represents a residue of carbohydrates selected from the group comprising monosaccharides, disaccharides, polysaccharides, polyols, as well as combinations of these residues,

X represents a C1-C12 aliphatic residue, linear or branched alkene, alkyne, or a unit of ethylene oxide or propylene oxide having a degree of polymerisation of between 1 and 10 as well as all combinations of these residues,

n=0 or 1

R1 represents H

R2, R3 independently represent a hydrogen atom or a linear or branched C1-C20 hydrocarbon chain or perfluorated, as well as all combinations of these substituents.

According to the present invention the amphiphilic transporter will be advantageously selected from amine surfactants with sugar head and long-chain, such as N-alkylamino-1-deoxylactitols comprising a chain with 12 or 16 carbon atoms which will respectively be called Lhyd12 and Lhyd16.

The general formula of N-alkylamino-1-deoxylactitols is the following:

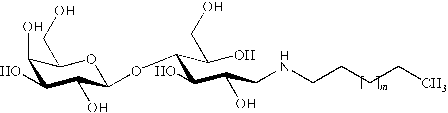

with m=8 for Lhyd12 and with m=12 for Lhyd16.

These derivatives are prepared according to one of the methods known from the prior art (New Journal of Chemistry, 1992, 16(3), 387; J. Dispersion Science and Technology, 1991, 12(3&4), 227.; Langmuir, 1999, 15, 6163.; Biochimica and biophysica acta, 1992, 1109, 55; FR2 661 413 publication of 31 Oct. 1991).

According to the present invention, the active principle will be preferably selected from non-steroidal anti-inflammatories (NSAI) carrying an acid function, such as ketoprofen, ibuprofen or indometacin. Likewise, the complexes according to the present invention could advantageously be utilised to solubilise and transport by acid/base association polyunsaturated fatty acids (PFA) such as linoleic acid or linolenic acid. In particular, according to the present invention, the intermolecular association (stoichiometric or not) will be formed by simple contact in water or other solvent, of the amphiphilic molecule in its basic form with the active principle in its acid form.

The invention therefore relates to an association by simple acid/base neutralisation between the amphiphilic transporter in its basic form and the active principle in its acid form.

The invention also relates to a process for preparation of the present complexes. For this purpose the stoichiometric mixture of amphiphilic transporter and active principle are advantageously made to react, by heating them at a temperature between ambient and boiling temperature of the solvent at atmospheric pressure, and over a period of 1 to 72 hours. After reaction water is removed from the final mixture, preferably filtered and lyophilised.

According to preferred embodiments of the invention, the reagents and solvent are selected as follows:
the basic transporter is Lhyd12 or Lhyd16
the active principle is indometacin, ibuprofen, ketoprofen or linoleic acid.
the solvent is water or methanol.

### EXAMPLE 1

Under magnetic agitation 138.9 mg (0.66 mmol) of ibuprofen are added to a solution of 337.7 mg (0.66 mmol) of Lhyd12 in 30 ml of distilled water maintained at 25° C. The mixture is maintained under agitation for 24 hours. After reaction, the aqueous solution containing the association is evaporated under pressure reduced at the pump to finally obtain 476.6 mg of product. The association constitutes a novel amphiphilic species which forms aggregates of 80 nm in diameter from CAC of 10⁻³M.

### EXAMPLE 2

Under magnetic agitation 171.3 mg (0.66 mmol) of ketoprofene are added to a solution of 337.7 mg (0.66 mmol) of Lhyd12 in 30 mL of distilled water maintained at 25° C. The mixture is maintained under agitation for 24 hours. After reaction, the aqueous solution containing the association is evaporated under pressure reduced at the pump to finally obtain 509 mg of product. The association constitutes a novel amphiphilic species which forms aggregates of 20 and 260 nm in diameter from CAC of 1.3 10⁻³ M.

### EXAMPLE 3

Under magnetic agitation 236.3 mg (0.66 mmol) of indometacin are added to a solution of 337.9 mg (0.66 mmol) of Lhyd12 in 30 mL of distilled water maintained at 25° C. The mixture is maintained under agitation for 24 hours. After reaction, the aqueous solution containing the association is lyophilised to finally obtain 574.2 mg of product.

The association constitutes a novel amphiphilic species which forms aggregates having a diameter of less than 10 nm from CAC of 10⁻³M.

### EXAMPLE 4

Under magnetic agitation 328.2 mg (0.91 mmol) of indometacin are added to a solution of 536.9 mg (0.91 mmol) of Lhyd16 in 42 mL of distilled water maintained at 25° C. The mixture is maintained under agitation for 24 hours. After reaction, the aqueous solution containing the association is lyophilised to finally obtain 865.1 mg of product.

The association constitutes a novel amphiphilic species which forms aggregates having a diameter of 50 nm from CAC of 4.5 10⁻⁴M.

### EXAMPLE 5

Transcutaneous penetration study conducted on two associations carried out using indometacin and Lhyd12 and Lhyd16.

The passage study is conducted ex vivo on pig ear skin in an infinite dose and with occlusion. The formulae tested are realised in aqueous solution, while indometacin control alone is prepared in hydroalcoholic gel (for solubility reasons).

The different formulae are the following:
formula A: preparation of indometacin at 2.5% of hydroalcoholic gel (EtOH/water 75/25)
formula B: preparation of an association at 2.5% of indometacin in aqueous solution
   formula B1: indometacin association associated with Lhyd16
   formula B2: indometacin association associated with Lhyd12
formula C: preparation of an association at 2.5% of indometacin in aqueous gel at 1.5% by viscosing
   formula C1: indometacin association associated with Lhyd16

The accumulated quantities of indometacin measured on completion of 24 hours are represented hereinbelow:

### EXAMPLE 6

469.7 mg of Lhyd12 (0.66 mmol) are solubilised in 30 ml of methanol. 208 μl (0.66 mmol) of linoleic acid taken under nitrogen are then introduced to the aqueous solution without particular precaution vis-à-vis air and light. The solution is maintained under agitation for 24 hours. The methanol is then evaporated under reduced pressure to result in gel which is absorbed in water and lyophilised to finally produce 655 mg of product. The association constitutes a novel amphiphilic species which forms two populations of aggregates of a diameter of 200 and 1000 nm from CAC of 2.5 10⁻²M.

### EXAMPLE 7

564.6 mg (0.96 mmol) of Lhyd16 are introduced in 42 ml of methanol. 302 μl (0.96 mmol) of linoleic acid taken under nitrogen are then introduced to the aqueous suspension without particular precaution vis-à-vis air and light. After 4 days of agitation at ambient temperature, the solution is evaporated under reduced pressure to result in gel which is absorbed in water and lyophilised to finally produce 833.6 mg of product.

1. An intermolecular association complex of formula (I) of an amphiphilic transporter and an active principle ⁻Z-Y: 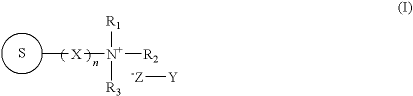
in which
S represents a residue of carbohydrates selected from the group comprising monosaccharides, disaccharides, polysaccharides, polyols, as well as combinations of these residues,
X represents a C1-C12 aliphatic residue selected from linear or branched alkyl; alkene, alkyne, or an ethylene oxide or propylene oxide unit having a degree of polymerisation of between 1 and 10 as well as all combinations of these residues,
n=0 or 1
R1 represents H
R2, R3 represents independently a hydrogen atom or a linear or branched C1-C20 hydrocarbon chain or perfluorated, as well as all combinations of these substituents, and in which the active principle
⁻Z-Y
comprises a residue
Y carrying the therapeutic or pro-therapeutic activity selected from the group comprising anti-inflammatories, antibiotics, polyunsaturated fatty chain, vitamins or pro-vitamins,
and a residue
Z acid selected from the group comprising carboxylates, sulphates, sulphonates, phosphates, phosphonates or phosphinates.

2. The intermolecular association complex as claimed in claim 1, wherein the amphiphilic transporter is selected from N-alkylamino-1-deoxylactitols of formula 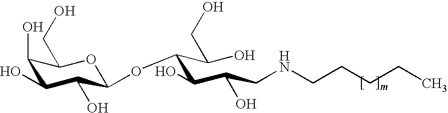
with m=8 or m=12.

3. The intermolecular association complex as claimed in claim 1, wherein the active principle is selected from indometacin, ibuprofen, ketoprofen or linoleic acid.

4. Use of a complex as claimed in any one of claims 1 to 3, to protect, solubilise and/or convey an active principle.

5. A drug intended for topical or transcutaneous administration comprising a complex as claimed in any one of claims 1 to 3.
